(19) United States
(12) Patent Application Publication
(10) Pub. No.: US 2024/0384222 A1
(43) Pub. Date: Nov. 21, 2024

(54) MANUFACTURING APPARATUS, INFORMATION PROCESSING APPARATUS, AND MANUFACTURING METHOD

(71) Applicants: Canon Medical Systems Corporation, Otawara-shi (JP); CANON KABUSHIKI KAISHA, Tokyo (JP)

(72) Inventors: Kanako AIZAWA, Yaita (JP); Kohei WATANABE, Utsunomiya (JP); Koji HIRATA, Kasukabe (JP); Mayumi SAKAGAMI, Ibaraki (JP)

(73) Assignees: Canon Medical Systems Corporation, Otawara-shi (JP); CANON KABUSHIKI KAISHA, Tokyo (JP)

(21) Appl. No.: 18/664,379

(22) Filed: May 15, 2024

(30) Foreign Application Priority Data
May 15, 2023 (JP) 2023-080000

(51) Int. Cl. C12M 1/36; C12M 1/00; C12M 1/34

(52) U.S. Cl. CPC C12M 41/48; C12M 29/06; C12M 41/30; C12M 41/46

(57) ABSTRACT

According to one embodiment, a manufacturing apparatus includes processing circuitry. The processing circuitry acquires information relating to proliferation characteristics of cells when using a cell suspension, determines a filling amount of the cell suspension corresponding to one housing container, based on the information relating to the proliferation characteristics, and fills the cell suspension of the filling amount into the housing container for containing the cell suspension of the filling amount.

### CROSS-REFERENCE TO RELATED APPLICATION

This application is based upon and claims the benefit of priority from prior Japanese Patent Application No. 2023-080000, filed May 15, 2023, the entire contents of which are incorporated herein by reference.

### FIELD

Embodiments described herein relate generally to a manufacturing apparatus, an information processing apparatus, and a manufacturing method.

### BACKGROUND

Conventionally, in the manufacture of a cell stock for keeping cells, the number of stored cells per container is uniformly determined in the case of the same kind of cells, regardless of the origin of cells (for example, a difference of donors and a difference of tissues) or proliferation characteristics (for example, a doubling time of cells). In a case of manufacturing a cell processing product by using the manufactured cell stock as a raw material, there is a case in which the number of cells after mass culture (expansion culture) for mass-producing cells varies, in particular, due to a variance of proliferation characteristics of cells.

For example, as regards a variance of proliferation characteristics of cells, there is case where proliferation of cells originating from a certain donor is slow, while proliferation of cells originating from another donor is fast. In addition, aside from the difference of donors, there is a case where the proliferation characteristics vary due to a difference of tissues from which the cells originate, or the like. Thus, in a case where the number of cells after expansion culture is less than a target number of cells, it is necessary to perform additional expansion culture, and an error may occur in a manufacturing schedule. On the other hand, in the case of cells of fast proliferation, passage that is not assumed at a time of expansion culture may become necessary, and additional work occurs. Besides, if passage becomes necessary at an earlier timing than assumed, the schedule needs to be adjusted. Thus, in any of the cases, it is possible that a cell stock manufactured without taking the proliferation characteristics of cells into account increases a work cost in a later process.

### BRIEF DESCRIPTION OF THE DRAWINGS

FIG. 1 is a diagram illustrating a configuration example of a manufacturing apparatus according to a first embodiment.

FIG. 2 illustrates a work flow exemplarily illustrating a user's work and an operation of the manufacturing apparatus in the first embodiment.

FIG. 3 is a flowchart exemplarily illustrating a concrete operation of the manufacturing apparatus in the first embodiment.

FIG. 4 is an explanatory view relating to culture in which proliferation characteristics of respective cells are taken into account.

FIG. 5 illustrates a work flow exemplarily illustrating a user's work in a second embodiment.

FIG. 6 is a flowchart exemplarily illustrating a supply method of a storing container of a manufacturing apparatus according to a third embodiment.

FIG. 7 is a flowchart exemplarily illustrating a supply method of a storing container of a manufacturing apparatus according to a fourth embodiment.

FIG. 8 is a flowchart exemplarily illustrating a supply method of a storing container of a manufacturing apparatus according to a fifth embodiment.

FIG. 9 is a diagram illustrating a configuration example of an information processing apparatus according to a sixth embodiment.

FIG. 10 is an explanatory view relating to culture in which proliferation characteristics of respective cells are not taken into account in conventional art.

### DETAILED DESCRIPTION

In general, according to one embodiment, a manufacturing apparatus includes processing circuitry. The processing circuitry acquires information relating to proliferation characteristics of cells when using a cell suspension, determines a filling amount of the cell suspension corresponding to one housing container, based on the information relating to the proliferation characteristics, and fills the cell suspension of the filling amount into the housing container for containing the cell suspension of the filling amount.

Hereinafter, referring to the accompanying drawings, embodiments of a manufacturing apparatus are described in detail. A manufacturing apparatus according to an embodiment relates to the manufacture of a cell-containing product in which a cell suspension including cultured cells is filled into a storing container. In the embodiments below, the manufacture of a cell stock in which a cell-containing product is filled into a storing container is mainly described. Note that, hereinafter, in a case where the term “culture” is simply described, it is assumed that “culture” means “expansion culture”, unless otherwise described. Also note that, in consideration of the possibility of the cell-containing product being used immediately after manufacture of the cell stock, the “storing container” may be utilized as a housing container for housing the cell suspension.

### First Embodiment

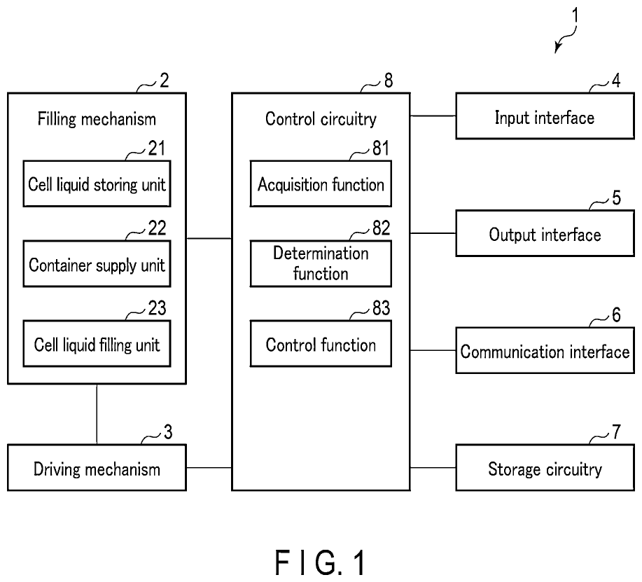

FIG. 1 is a diagram illustrating a configuration example of a manufacturing apparatus 1 according to a first embodiment. The manufacturing apparatus 1 includes a filling mechanism 2, a driving mechanism 3, an input interface 4, an output interface 5, a communication interface 6, storage circuitry 7, and control circuitry 8.

The filling mechanism 2 stores a cell suspension that is supplied from the outside of the manufacturing apparatus 1, and fills the stored cell suspension into a storing container corresponding to a filling amount. Specifically, the filling mechanism 2 includes a cell liquid storing unit 21 (storing unit), a container supply unit 22 (supply unit), and a cell liquid filling unit 23 (filling unit).

The cell liquid storing unit 21 is a container that stores a cell suspension that is supplied from the outside of the manufacturing apparatus 1. The cell liquid storing unit 21 includes a stirring unit such as a rotator, and can stir the cell suspension. In addition, the cell liquid storing unit 21 is connected to the cell liquid filling unit 23 via a conduit such as a resin-made tube. Note that the filling mechanism 2 may include a stirrer under the cell liquid storing unit 21 in order to drive the rotator.

The container supply unit 22 supplies a storing container corresponding to a filling amount of the cell suspension, in accordance with control of the control circuitry 8. Methods of supplying a storing container include fabrication of a storing container, capacity adjustment of a variable storing container (variable container), and selection of an optimal storing container from among a plurality of different storing containers. Concrete descriptions thereof are given in embodiments below. Note that the container supply unit 22 may present an appropriate storing container to a user, and the user may acquire the storing container.

In accordance with control of the control circuitry 8, the cell liquid filling unit 23 fills the cell suspension stored in the cell liquid storing unit 21 into the storing container supplied by the container supply unit 22. The cell liquid filling unit 23 includes a pump and an on-off valve, and fills a cell suspension of a filling amount according to an instruction of the control circuitry 8 from the cell liquid storing unit 21 into the storing container. Note that the cell liquid filling unit 23 may be configured to enable the user to manually install the acquired storing container.

Note that the filling mechanism 2 may include a take-out port for outputting (taking out) the storing container filled with the cell suspension.

The driving mechanism 3 drives the filling mechanism 2 in accordance with control of the control circuitry 8. The driving mechanism 3 is implemented by, for example, a gear, a stepping motor, a belt conveyor, and a lead screw. For example, the driving mechanism 3 drives the stirrer of the cell liquid storing unit 21, the container supply unit 22, and the cell liquid filling unit 23.

The input interface 4 accepts, by an operation of an operator (user), setting of a target condition including a culture period of cells (cell culture period) and a target number of cells (target cell number). The input interface 4 is implemented by, for example, a mouse, a keyboard, and a touch pad to which an instruction is input by touching on an operation surface thereof. The input interface 4 is connected to the control circuitry 8, converts an operation instruction, which is input by the user, into an electric signal, and outputs the electric signal to the control circuitry 8.

In the present specification, the input interface 4 is not limited to an input interface including physical operation components such as a mouse and a keyboard. For example, the input interface 4 may be processing circuitry that receives an electric signal corresponding to an operation instruction that is input from an external input device provided separately from the manufacturing apparatus 1, and outputs the electric signal to the control circuitry 8.

The output interface 5 is connected to the control circuitry 8, and outputs a signal that is input from the control circuitry 8. The output interface 5 is implemented by, for example, display circuitry, printing circuitry, and an audio device.

The display circuitry includes, for example, a CRT display, a liquid crystal display, an organic EL display, an LED display, and a plasma display. In addition, the display circuitry may be processing circuitry that converts data representing a display target into a video signal, and outputs the video signal to the outside. The printing circuitry includes, for example, a printer. In addition, the printing circuitry may be processing circuitry that outputs data representing a display target to the outside. The audio device includes, for example, a speaker. In addition, the audio device may be processing circuitry that outputs an audio signal to the outside.

The communication interface 6 is connected to, for example, a server. In accordance with control of the control circuitry 8, the communication interface 6 receives, from the server, information (cell information) relating to cells for the manufacture of a cell stock. The cell information includes, for example, information relating to proliferation characteristics of cells when using a cell suspension (e.g., during culture of the cells) (proliferation characteristic information), and information relating to a cell suspension (suspension information).

The proliferation characteristic information includes, for example, at least one of a proliferation rate or a doubling time of cells, and a proliferation curve of cells. For example, the proliferation characteristic information may include sample information (e.g., an age, a gender, and a biomarker) on a sample from which the cells originate. The suspension information includes, for example, at least one of a total amount of a cell suspension, and a living cell number and a cell concentration in a cell suspension.

The storage circuitry 7 includes, for example, a magnetic recording medium, an optical recording medium, or a recording medium that is readable by a processor, such as a semiconductor memory. Note that the storage circuitry 7 is not always implemented by a single storage device. For example, the storage circuitry 7 may be implemented by a plurality of storage devices.

In addition, the storage circuitry 7 stores, for example, the target condition that is input from the user, cell information acquired by the communication interface 6 via the server, and patient information acquired by the communication interface 6 via an intra-hospital network NW. In addition, the storage circuitry 7 stores an operation program that is read out by the control circuitry 8.

The control circuitry 8 is a processor functioning as a central unit of the manufacturing apparatus 1. For example, the control circuitry 8 outputs control signals for driving the respective units of the filling mechanism 2 to the driving mechanism 3. Note that the control circuitry 8 may include a memory that stores at least a part of the data stored in the storage circuitry 7.

In addition, by executing an operation program stored in the storage circuitry 7, the control circuitry 8 implements functions corresponding to this operation program. Specifically, the control circuitry 8 includes an acquisition function 81 (acquisition unit), a determination function 82 (determination unit) and a control function 83 (control unit) by executing the operation program. Note that in the present embodiment, a case is described in which the acquisition function 81, determination function 82 and control function 83 are implemented by a single processor, but the embodiment is not limited to this. For example, a plurality of independent processors may be combined to constitute control circuitry, and the respective processors may execute the operation program, thereby implementing the acquisition function 81, determination function 82 and control function 83.

By the acquisition function 81, the control circuitry 8 acquires the cell information. Specifically, the control circuitry 8 acquires the proliferation characteristic information at a time of cell culture of cells included in a cell suspension. In addition, the control circuitry 8 may acquire the suspension information and the target condition.

By the determination function 82, the control circuitry 8 determines, based on the proliferation characteristic information, a filling amount of the cell suspension corresponding to one cell stock. Specifically, for example, in a case where the proliferation characteristic information includes the proliferation rate of cells, the control circuitry 8 determines the filling amount, based on the proliferation rate. In a case where the proliferation characteristic information includes the doubling time of cells, the control circuitry 8 may determine the filling amount, based on the doubling time. In a case where the proliferation characteristic information includes the proliferation curve of cells, the control circuitry 8 may determine the filling amount, based on the proliferation curve. Note that the control circuitry 8 may determine a plurality of filling amounts, and may use the determined filling amounts as candidates for the filling amount.

Besides, by the determination function 82, the control circuitry 8 may determine the filling amount, based on the proliferation characteristic information, suspension information, and target condition. Specifically, for example, in a case where the proliferation characteristic information includes the proliferation rate of cells, the control circuitry 8 may determine the filling amount, based on the proliferation rate, suspension information, and target condition. In addition, in a case where the proliferation characteristic information includes the proliferation curve of cells, the control circuitry 8 may determine the filling amount, based on the proliferation curve, suspension information, and target condition. Furthermore, in a case where the proliferation characteristic information includes the doubling time of cells, the control circuitry 8 may determine the filling amount, based on the doubling time, suspension information, and target condition.

By the control function 83, the control circuitry 8 controls the operations of the respective components of the manufacturing apparatus 1. Specifically, the control circuitry 8 causes the container supply unit 22 to supply a storing container for containing a cell suspension of the determined filling amount. In addition, the control circuitry 8 causes the cell liquid filling unit 23 to fill a cell suspension of the determined filling amount into the supplied storing container.

Note that by the control function 83, the control circuitry 8 may present a determined filling amount, or candidate filling amounts, including a plurality of filling amounts, on a display functioning as the output interface 5. In addition, by the control function 83, the control circuitry 8 may determine whether an instruction from the user was accepted. Besides, by the control function 83, the control circuitry 8 may output (discharge) the storing container, into which the cell suspension is filled, from the take-out port of the filling mechanism 2.

The configuration of the manufacturing apparatus 1 according to the first embodiment has been described above. Next, a user's work and an operation of the manufacturing apparatus in the first embodiment are described.

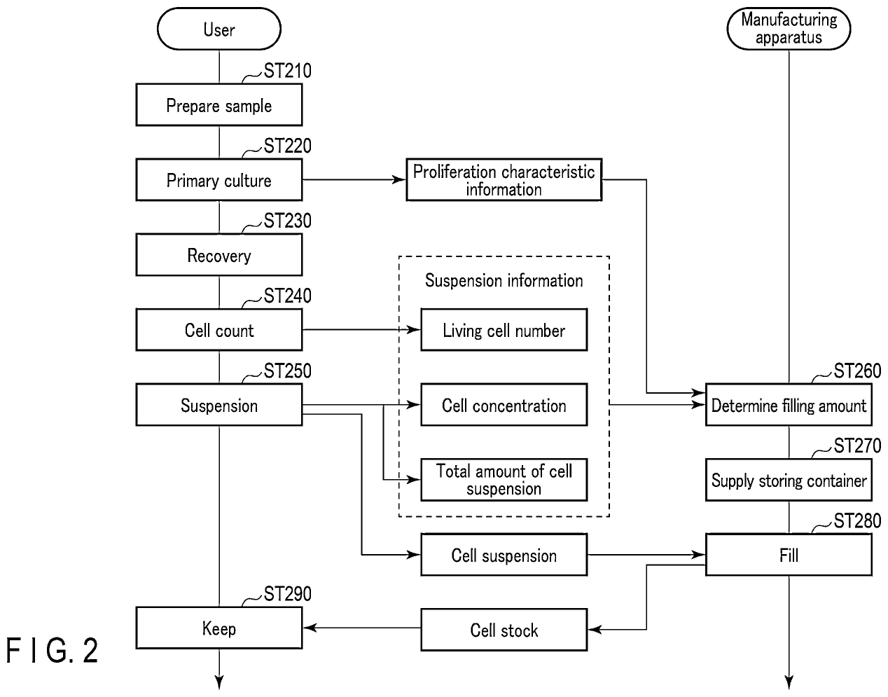

FIG. 2 illustrates a work flow exemplarily illustrating the user's work and the operation of the manufacturing apparatus in the first embodiment. Hereinafter, referring to the work flow of FIG. 2, a serial flow relating to cell stock manufacture is described. Note that a description of details of each of steps of the user's work is omitted since freely selected methods may be used for the steps.

### (Step ST210)

To begin with, the user prepares a sample. Specifically, the user samples blood or tissue from a donor. The user isolates cells to be cultured, from the sampled blood or tissue. After isolating the cells, the user prepares for the start of culture by cell seeding and cell observation.

Note that in a case of using cells of a cell line, since most of them are frozen, the user prepares for the start of culture after performing a process of unfreezing the frozen cells.

### (Step ST220)

After performing the preparation for the start of culture, the user cultures cells (primary culture). In the present embodiment, the primary culture means culturing cells whose proliferation characteristic information is unknown. Specifically, the user prepares a culture container corresponding to the type of cells (for example, adherent cells, semi-adherent cells, and floating cells), and performs culture. A cell culture period is freely determined by the user. Information acquired at a time of cell culture is managed, for example, by a server as proliferation characteristic information. Note that in the primary culture, passage may be performed.

The proliferation characteristic information is, for example, the number of seed cells at the time of starting primary culture, a culture time up to a freely selected time point during the primary culture and the number of cells at this time point, and a proliferation rate or a doubling time of cells, and a proliferation curve of cells, which are calculated based on a culture time up to an end time of the primary culture and the number of cells at the end time point.

### (Step ST230)

After the end of the primary culture, the user recovers the cultured cells from the culture container. In a case where the cultured cells are adherent cells or semi-adherent cells, the user peels the cells adhering to the culture container or the like, and then recovers the cells.

### (Step ST240)

After recovering the cells, the user performs a cell count of the recovered cells. Examples of the cell count include a manual count by a hemocytometer, and an automatic count based on flow cytometry and microscopic images. Information acquired at the time of the cell count is managed, for example, by a server as a living cell number that is the total number of cultured cells.

### (Step ST250)

After performing the cell count, the user prepares a cell suspension by using a cell storing liquid of an amount corresponding to a freely selected cell concentration. Information acquired at the time of preparing the cell suspension is managed, for example, by the server as a cell concentration that is the ratio (concentration) of the number of cells included in the prepared cell suspension, and a cell suspension total amount that is the total amount of the prepared cell suspension. The user supplies the prepared cell suspension to the manufacturing apparatus 1.

### (Step ST260)

After the proliferation characteristic information is managed by the server in step ST220, the manufacturing apparatus 1 determines, based on the proliferation characteristic information, a filling amount of the cell suspension corresponding to one cell stock. However, since there is a case where necessary information is deficient in order to determine the filling amount by using only the proliferation characteristic information, the manufacturing apparatus 1 may determine the filling amount, based on the proliferation characteristic information and the suspension information, after the suspension information (living cell number, cell concentration, and cell suspension total amount) is managed by the server in step ST240 and step ST250. In addition, the manufacturing apparatus 1 may determine the filling amount, further based on the target condition (cell culture period and target cell number) that is input by the user. Hereinafter, the determination of the filling amount is described by taking a concrete example.

For example, a case is assumed in which, as regards certain cells, primary culture was conducted with the seed cell number at the culture start time being 0.5×10∧6, and the cell culture period being 120 hours, and, as a result, the number of the recovered living cells was 4×10∧6. In this case, since the cell number increases eight times in 120 hours, the cell doubling time is 40 hours.

In the case of further performing the culture by using the cells, if it is assumed that the target cell number is 4×10∧6 and the cell culture period is 120 hours, the seed cell number at the culture start time becomes 0.5×10∧6. In addition, if it is assumed that the concentration of the cell suspension is 0.5×10∧6/mL, a cell suspension of a filling amount of 1.0 mL is filled into the storing container corresponding to one cell stock.

Next, a case is considered in which, as regards cells of three lines (cells A, cells B and cells C) of different proliferation characteristics, cell stocks thereof are prepared, and the same cell culture period (for example, 120 hours) and target cell number (for example, 4×10∧6) are set. In a case where the cell doubling times of the cells of these three lines are 30 hours, 40 hours and 60 hours, respectively, the seed cell numbers at the culture start time become 0.25×10∧6, 0.5×10∧6, and 1.0×10∧6, respectively. In addition, if it is assumed that the concentration of the cell suspension is constant at 0.5×10∧6/mL, cell suspensions of filling amounts of 0.5 mL, 1.0 mL and 2.0 mL are filled into storing containers each corresponding to one cell stock.

Since it is considered that the actual proliferation ability (proliferation characteristic) is affected by the cell density at the time of culture, it is considered desirable to start expansion culture within the range of the cell density (seed density) with a track record at the time of seeding. Specifically, it is considered desirable that the seed density using the filling cell number of the cell stock is set to be not lower than the cell density at the time of seeding in the culture period before the manufacture of the cell stock and at the data acquisition time of the cell number. For example, it is possible that, as regards the above-described cells A (the cell doubling time is 30 hours), the cell density at the time of seeding is examined, and, as a result, the filling cell number per storing container is determined to be a cell number larger than 0.25×10∧6. Hence, the seed density may be included in the proliferation characteristic information.

Furthermore, a cell morphology may be included as the proliferation characteristic information. The cell morphology is acquired, for example, by photographing cells that are being cultured, and image-analyzing the photographed images.

### (Step ST270)

After determining the filling amount of the cell suspension corresponding to one cell stock, the manufacturing apparatus 1 supplies a storing container for containing the cell suspension of the filling amount.

### (Step ST280)

After supplying the storing container, the manufacturing apparatus 1 fills the cell suspension of the determined filling amount into the supplied storing container. The manufacturing apparatus 1 outputs a plurality of storing containers, into which the cell suspension is filled, as cell stocks.

### (Step ST290)

After the cells stocks are output from the manufacturing apparatus 1, the user keeps the cell stocks by a freely selected method. Typically, the cell stocks are frozen and kept.

Note that in step ST220, step ST240 and step ST250, the cell information (proliferation characteristic information and suspension information) was described as being managed by the server, but the embodiment is not limited to this. For example, the cell information may be managed by the storage circuitry 7 of the manufacturing apparatus 1.

The user's work and the operation of the manufacturing apparatus in the first embodiment have been described above. Next, a concrete operation of the manufacturing apparatus according to the first embodiment is described

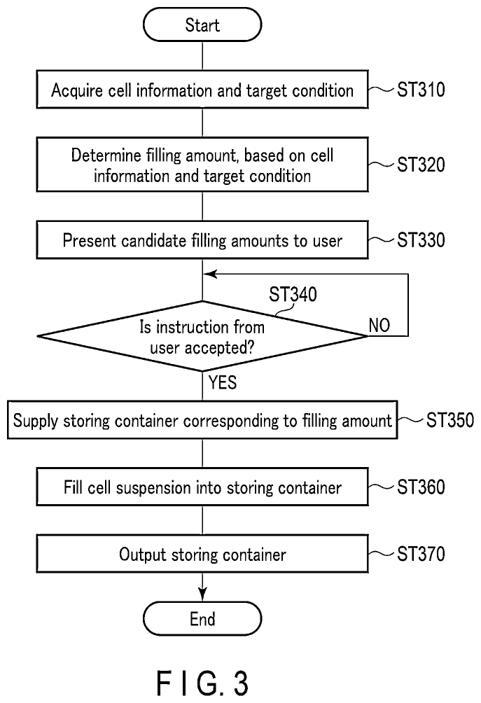

FIG. 3 is a flowchart exemplarily illustrating a concrete operation of the manufacturing apparatus according to the first embodiment. It is assumed that at a start time of the flowchart of FIG. 3, a cell suspension to be filled into the storing container is stored in the cell liquid storing unit 21.

### (Step ST310)

The control circuitry 8 executes the acquisition function 81. Upon executing the acquisition function 81, the control circuitry 8 acquires the cell information and target condition.

### (Step ST320)

After acquiring the cell information and target condition, the control circuitry 8 executes the determination function 82. Upon executing the determination function 82, the control circuitry 8 determines a filling amount, based on the cell information and target condition.

### (Step ST330)

After determining the filling amount, the control circuitry 8 executes the control function 83. Upon executing the control function 83, the control circuitry 8 presents candidate filling amounts to the user. Specifically, the control circuitry 8 causes the display functioning as the output interface 5 to display the candidate filling amounts.

### (Step ST340)

After displaying the candidate filling amounts, the control circuitry 8 determines, by the control function 83, whether an instruction from the user is accepted. The instruction from the user is an instruction for selecting the filling amount from among the presented candidate filling amounts. If the instruction from the user is accepted, the process advances to step ST350. If the instruction from the user is not accepted, the process stands by in step ST340 until accepting an instruction from the user.

### (Step ST350)

After accepting the instruction from the user, the control circuitry 8 causes, by the control function 83, the container supply unit 22 to supply a storing container corresponding to the filling amount. At this time, the container supply unit 22 supplies the storing container corresponding to the filling amount.

### (Step ST360)

After supplying the storing container, the control circuitry 8 causes, by the control function 83, the cell liquid filling unit 23 to fill the cell suspension into the storing container. At this time, the cell liquid filling unit 23 fills the cell suspension of the determined filling amount into the storing container.

### (Step ST370)

After filling the cell suspension into the storing container, the control circuitry 8 causes, by the control function 83, the filling mechanism 2 to output the storing container filled with the cell suspension. At this time, the filling mechanism 2 outputs the storing container filled with the cell suspension. After step ST370, the process of the flowchart of FIG. 3 ends.

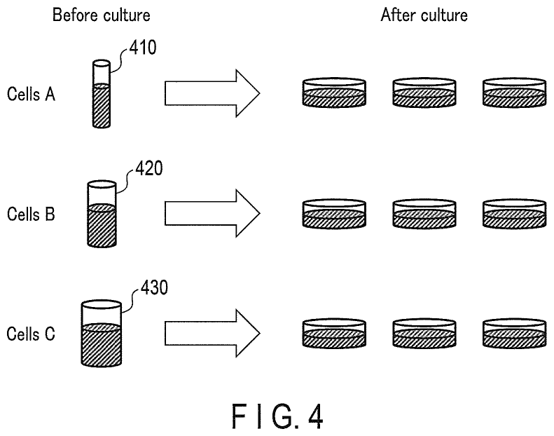

The concrete operation of the manufacturing apparatus according to the first embodiment has been described above. Next, referring to FIG. 10 and FIG. 4, a description is given of conventional culture in which proliferation characteristics are not taken into account, and culture in the first embodiment in which proliferation characteristics are taken into account.

FIG. 10 is an explanatory view relating to culture in which proliferation characteristics of respective cells are not taken into account in conventional art. FIG. 10 illustrates states before cell culture and states after cell culture in regard to the above-described cells A, cells B and cells C having different proliferation characteristics.

In a storing container 1010 relating to the cells A, for example, a cell suspension in an amount of 1.0 mL including the cells A is filled. For example, in a storing container 1020 relating to the cells B and a storing container 1030 relating to the cells C, a cell suspension in an amount of 1.0 mL including the cells B and a cell suspension in an amount of 1.0 mL including the cells C are filled.

The cells A, cells B and cells C have different proliferation characteristics. Thus, the living cell numbers after cell culture are different therebetween. FIG. 10 illustrates that culture for four culture containers was conducted in regard to the cells A, culture for three culture containers was conducted in regard to the cells B, and culture for two culture containers was conducted in regard to the cells C. If the target condition is set to be three culture containers, one culture container is surplus in regard to the cells A, and one culture container is deficient in regard to the cells C.

FIG. 4 is an explanatory view relating to culture in which proliferation characteristics of respective cells are taken into account. FIG. 4 illustrates states before cell culture and states after cell culture in regard to the above-described cells A, cells B and cells C having different proliferation characteristics.

In a storing container 410 relating to the cells A, for example, a cell suspension in an amount of 0.5 mL including the cells A is filled. In addition, in a storing container 420 relating to the cells B, for example, a cell suspension in an amount of 1.0 mL including the cells B is filled. Further, in a storing container 430 relating to the cells C, for example, a cell suspension in an amount of 2.0 mL including the cells C is filled.

The storing container 410, storing container 420 and storing container 430 have, for example, sizes corresponding to filling amounts. For example, the volume of the storing container 410 is less than the volume of the storing container 420. In addition, the volume of the storing container 420 is less than the volume of the storing container 430.

The cells A, cells B and cells C have mutually different proliferation characteristics. Thus, in the present embodiment, the filling amounts (cell numbers) before cell culture are made different between the respective cells. Thereby, even in the case of the cells having different proliferation characteristics, the living cell numbers after cell culture can be made substantially equal. FIG. 4 illustrates the case in which the culture for three culture containers was conducted in regard to the respective cells.

As has been described above, the manufacturing apparatus according to the first embodiment acquires information relating to proliferation characteristics at a time of cell culture of cells included in a cell suspension, determines a filling amount of the cell suspension corresponding to one cell stock, based on the information relating to the proliferation characteristics, and fills the cell suspension of the filling amount into a storing container for containing the cell suspension of the filling amount.

Accordingly, the manufacturing apparatus according to the first embodiment can make a cell number after expansion culture substantially equal to a target cell number, by considering a variance in proliferation characteristics, in regard to the cell suspension that is filled into the cell stock. Thus, the manufacturing apparatus according to the first embodiment can manufacture a cell stock in which proliferation characteristics of cells are taken into account.

For example, in a case where cell stocks manufactured by the present manufacturing apparatus are used for expansion culture, the expansion culture of cells having a high proliferation ability can be started from a state in which the cell number is small, and the expansion culture of cells having a low proliferation ability can be started from a state in which the cell number is large. Hence, after a predetermined expansion culture period, it is expected that the cell numbers of both cells reach a substantially equal cell number. In other words, it is assumed that determination standards for target cell numbers are cleared at the same timing. Thereby, a variance after culture can be reduced, and it is considered that the management relating to the plan and execution of a manufacturing schedule becomes easier.

### Second Embodiment

In the first embodiment, the case was mainly described in which the proliferation characteristic information at the time of cell culture is acquired by using cells sampled from a donor. On the other hand, in a second embodiment, a description is given of a case where proliferation characteristic information, in which a process of freezing and unfreezing is also taken into account, is acquired in regard to the cells sampled from the donor.

It is assumed that a cell stock manufactured from cells sampled from a donor is frozen and kept. However, the influence by the process of freezing and unfreezing is not taken into account in the proliferation characteristic information acquired by primary culture. Thus, it is considered useful to once freeze and unfreeze the cells sampled from the donor, and then to culture the cells and acquire proliferation characteristic information. Hereinafter, information relating to proliferation characteristics at a time of cell culture of cells, for which the process of freezing and unfreezing is not executed, is referred to as “first proliferation characteristic information.” In addition, information relating to proliferation characteristics at a time of cell culture of cells, for which the process of freezing and unfreezing is executed, is referred to as “second proliferation characteristic information.” Note that the proliferation characteristic information may include the first proliferation characteristic information and the second proliferation characteristic information.

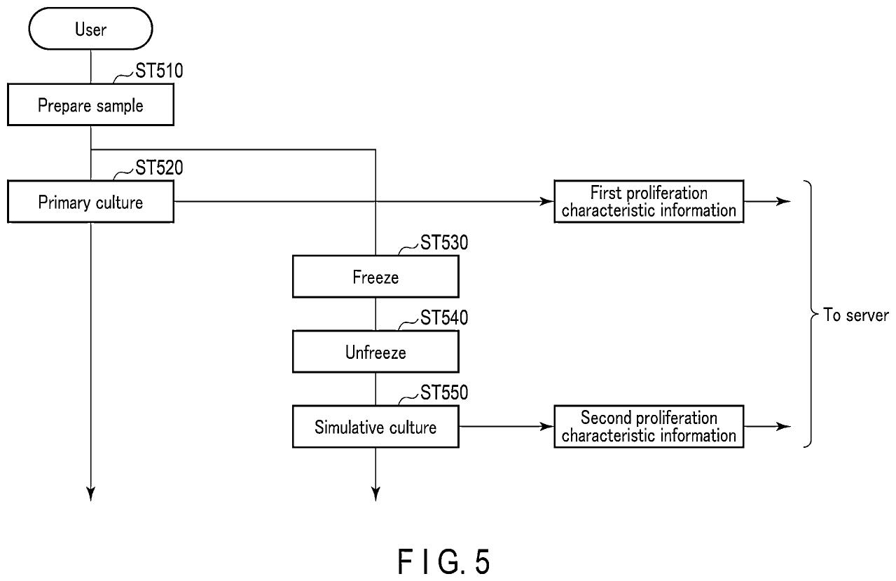

FIG. 5 illustrates a work flow exemplarily illustrating a user's work in the second embodiment. The work flow of FIG. 5 corresponds to a work flow in which step ST210 and step ST220 of the work flow of FIG. 2 are replaced. In the work flow of FIG. 5, the illustration of the steps after the recovery of cells in the user's work (for example, step ST230 onwards in FIG. 2) and the operation of the manufacturing apparatus is omitted.

### (Step ST510)

To begin with, the user prepares a sample. In the present embodiment, it is assumed that blood or tissue sampled from a donor is used. Thus, the user isolates cells to be used for culture, from the sampled blood or tissue. After isolating the cells, the user prepares for the start of culture by cell seeding and cell observation, in regard to the cells for primary culture and the cells for simulative culture. In the present embodiment, the simulative culture means culture of cells after the process of freezing and unfreezing the cells.

### (Step ST520)

After performing the preparation for the start of culture, the user cultures cells (primary culture). Specifically, the user prepares a culture container corresponding to the type of cells (for example, adherent cells, semi-adherent cells, and floating cells), and performs culture. Information acquired at a time of primary culture is managed, for example, by a server as the first proliferation characteristic information.

### (Step ST530)

In parallel with step ST520, the user freezes the cells for the simulative culture. This step simulates the keeping of the frozen cell stock in step ST290 of FIG. 2.

### (Step ST540)

After freezing the cells, the user unfreezes the frozen cells.

### (Step ST550)

After unfreezing the cells, the user cultures the cells (simulative culture). Since this step is substantially the primary culture, this step is similar to step ST520. Information acquired at the time of the simulative culture is managed, for example, by the server as the second proliferation characteristic information.

Note that after the second proliferation characteristic information is managed by the server in step ST550, the manufacturing apparatus 1 (not illustrated) determines the filling amount of the cell suspension corresponding to one cell stock, based on the first proliferation characteristic information and the second proliferation characteristic information.

Specifically, in a case where the proliferation rate of cells included in the second proliferation characteristic information is lower than the proliferation rate of cells included in the first proliferation characteristic information, the manufacturing apparatus 1 may determine a first filling amount based on the first proliferation characteristic information and a second filling amount based on the second proliferation characteristic information. In this case, the second filling amount is larger than the first filling amount. In addition, the manufacturing apparatus 1 may present candidates for the determined filling amounts to the user and may accept an instruction as to which of the filling amounts is selected.

Furthermore, it is desirable to set the same culture conditions (the composition of a culture medium, concentration, temperature, humidity, and the kind of culture container) for the primary culture and the simulative culture. By setting the same culture conditions, it is considered that the influence due to factors other than proliferation characteristics can be reduced.

From the above, in the second embodiment, the control circuitry 8 determines, by the determination function 82, the filling amount of the cell suspension corresponding to one cell stock, based on the first proliferation characteristic information and the second proliferation characteristic information. In other words, the proliferation characteristic information includes the first proliferation characteristic information at the time of primary culture and the second proliferation characteristic information at the time of simulative culture, and the control circuitry 8 determines the filling amount, based on the first proliferation characteristic information and second proliferation characteristic information.

As has been described above, the manufacturing apparatus according to the second embodiment acquires information relating to proliferation characteristics at a time of cell culture of cells included in a cell suspension, determines a filling amount of the cell suspension corresponding to one cell stock, based on the information relating to the proliferation characteristics, and fills the cell suspension of the filling amount into a storing container for containing the cell suspension of the filling amount. In the second embodiment, the information relating to proliferation characteristics includes the first proliferation characteristic information at the time of primary culture of cells and the second proliferation characteristic information at the time of simulative culture after the freezing and unfreezing of the cells, and the present manufacturing apparatus determines the filling amount, based on the first proliferation characteristic information and second proliferation characteristic information.

Accordingly, the manufacturing apparatus according to the second embodiment can manufacture a cell stock in which the influence of proliferation characteristics due to the freezing and unfreezing of the cell suspension is taken into account.

### Another Example of the Second Embodiment

In the second embodiment, the case was described in which the primary culture and simulative culture are performed for the cells sampled from the donor. On the other hand, in another example of the second embodiment, cells to be used in simulative culture may be cells after primary culture. Hereinafter, a description is given by referring to the flowchart of FIG. 5.

In the another example of the second embodiment, in step ST530, the cells sampled from the donor are not frozen, but the cells after primary culture are frozen. Specifically, in the another example of the second embodiment, the cells used in the simulative culture are the cells after the primary culture. In the manufacture of a cell stock, cells after primary culture are basically frozen and kept, and thus to perform the simulative culture after freezing and unfreezing the cells after the primary culture reflects the actual use environment. Therefore, it is expected that the proliferation characteristic information acquired in the another example of the second embodiment has a higher degree of certainty than the proliferation characteristic information acquired in the second embodiment.

### Third Embodiment

In the first embodiment and the second embodiment, it was described that a freely chosen method is used for supplying a storing container by the container supply unit 22. In a third embodiment, a description is given of a method of fabricating a storing container as a concrete supply method of the storing container by the container supply unit 22.

In the third embodiment, by the control function 83, the control circuitry 8 selects a mold corresponding to a filling amount. After selecting the mold, the control circuitry 8 causes the container supply unit 22 to fabricate a storing container by using the selected mold. In addition, the control circuitry 8 causes the container supply unit 22 to perform a sterilization process or particle removal for the fabricated storing container. Further, the control circuitry 8 causes the container supply unit 22 to output the storing container corresponding to the filling amount.

In the third embodiment, the container supply unit 22 is, for example, a blow molding machine. In accordance with control of the control circuitry 8, the container supply unit 22 fabricates a storing container by using the selected mold. Specifically, the container supply unit 22 fabricates the storing container, for example, by blow molding in which a molten resin is put in a mold and compressed air is blown into the inside of the resin. In addition, in accordance with control of the control circuitry 8, the container supply unit 22 performs a sterilization process or particle removal for the fabricated storing container. Further, in accordance with control of the control circuitry 8, the container supply unit 22 outputs the storing container corresponding to the filling amount.

Note that in the third embodiment, the container supply unit 22 may include a mold necessary for fabricating the storing container, and a resin serving as a raw material of the storing container. In addition, the container supply unit 22 may fabricate the storing container by methods other than the blow molding. Besides, the container supply unit 22 may fabricate the storing container corresponding to the filling amount by methods other than the fabrication of the storing container using the mold and the resin.

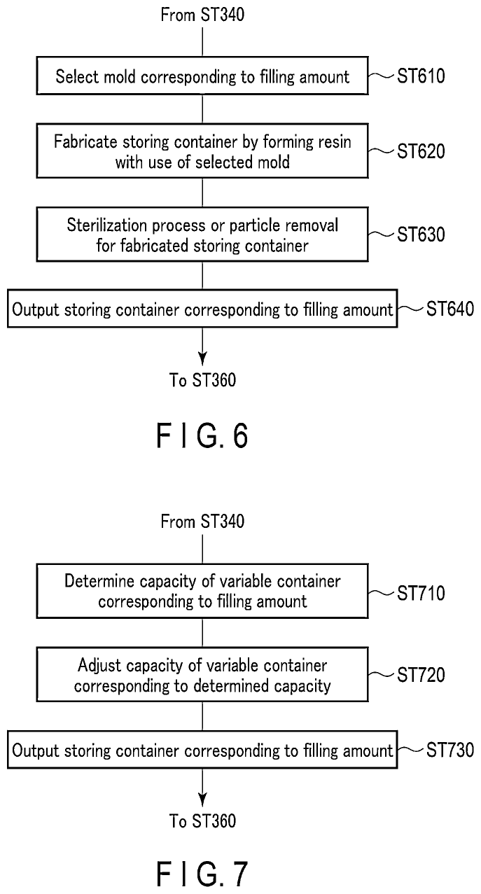

FIG. 6 is a flowchart exemplarily illustrating a supply method of a storing container of a manufacturing apparatus according to the third embodiment. The flowchart of FIG. 6 is a concrete example of step ST350 of the flowchart of FIG. 3. The flowchart of FIG. 6 starts after the process of step ST340 of FIG. 3.

### (Step ST610)

After accepting an instruction from the user in step ST340, the control circuitry 8 selects a mold corresponding the filling amount by the control function 83. Specifically, the control circuitry 8 selects the mold corresponding the filling amount, based on information in which a plurality of molds included in the container supply unit 22 and filling amounts corresponding to the molds are correlated.

### (Step ST620)

After selecting the mold, the control circuitry 8 outputs information of the mold to the container supply unit 22. Using the selected mold, the container supply unit 22 fabricates the storing container. Specifically, the container supply unit 22 fabricates the storing container by blow-molding the resin in the mold.

### (Step ST630)

After fabricating the storing container, the container supply unit 22 performs the sterilization process or the particle removal for the fabricated storing container. Note that the container supply unit 22 may perform both the sterilization process and the particle removal for the fabricated storing container, or may perform neither of them if not necessary.

### (Step ST640)

After performing the sterilization process or the particle removal for the storing container, the container supply unit 22 outputs the storing container corresponding to the filling amount. After step ST640, the process advances to step ST360 of FIG. 3.

As has been described above, the manufacturing apparatus according to the third embodiment fabricates the storing container corresponding to the filling amount, at the time of supplying the storing container.

Accordingly, since the manufacturing apparatus according to the third embodiment can fabricate an optimal storing container corresponding to the filling amount, an optimal cell stock can be manufactured for each of lots or for each of donors.

### Fourth Embodiment

In the third embodiment, the method of fabricating the storing container was described as a concrete supply method of the storing container by the container supply unit 22. In a fourth embodiment, a method of adjusting the capacity of a variable container is described as a concrete supply method of the storing container by the container supply unit 22.

In the fourth embodiment, by the control function 83, the control circuitry 8 determines the capacity of a variable container in accordance with a filling amount. After determining the capacity of the variable container, the control circuitry 8 causes the container supply unit 22 to adjust the capacity of the variable container in accordance with the determined capacity. In addition, the control circuitry 8 causes the container supply unit 22 to output the storing container corresponding to the filling amount.

In the fourth embodiment, in accordance with control of the control circuitry 8, the container supply unit 22 adjusts the capacity of the variable container in accordance with the determined capacity. Specifically, the container supply unit 22 adjusts the capacity of the variable container, for example, by melt-adhering a part of a sterilized cell storing bag serving as a storing container by a sealer or the like. In addition, the container supply unit 22 outputs the storing container corresponding to the filling amount in accordance with control of the control circuitry 8.

Note that in the fourth embodiment, the container supply unit 22 may include a sealer that is necessary for capacity adjustment of the variable container.

FIG. 7 is a flowchart exemplarily illustrating a supply method of a storing container of a manufacturing apparatus according to the fourth embodiment. The flowchart of FIG. 7 is a concrete example of step ST350 of the flowchart of FIG. 3. The flowchart of FIG. 7 starts after the process of step ST340 of FIG. 3.

### (Step ST710)

After accepting an instruction from the user in step ST340, the control circuitry 8 determines, by the control function 83, the capacity of the variable container in accordance with the filling amount.

### (Step ST720)

After determining the capacity of the variable container, the control circuitry 8 outputs the information of the capacity of the variable container to the container supply unit 22. The container supply unit 22 adjusts the capacity of the variable container in accordance with the determined capacity. Specifically, the container supply unit 22 adjusts the capacity of the variable container by melt-adhering a part of a cell storing bag by a sealer or the like.

For example, in a case of filling 10 mL of a cell suspension into a cell storing bag that can be filled with 20 ml of the cell suspension in a state of 100% of the flat plate area, the container supply unit 22 adjusts the capacity of the cell storing bag by melt-adhering a region of 50% of the plat plate area. By the capacity adjustment, the thicknesses of cell storing bags filled with the cell suspension can be made substantially equal. In the case of not performing the capacity adjustment, the thickness of the cell storing bag becomes different if the filling amount of the cell suspension is different. If the thickness of the cell storing bag is different, there is a possibility that an adverse effect occurs on a survival rate of cells included in the cell suspension that is filled, at a time of freezing and keeping the cell storing bag. In order to avoid such a possibility, it is considered useful to make equal the thicknesses of cell storing bags, even if the filling amounts of the cell suspension are different.

In addition, it is considered desirable for the container supply unit 22 to melt-adhere a peripheral part of the cell storing bag. By melt-adhering the peripheral part of the cell storing bag, the cell suspension can be filled in a central part of the cell storing bag, and it can thus be expected that the cell suspension that is filled can be protected from an external impact.

### (Step ST730)

After adjusting the capacity of the variable container, the container supply unit 22 outputs the storing container corresponding to the filling amount. After step ST730, the process advances to step ST360 of FIG. 3.

As has been described above, at the time of supplying the storing container, the manufacturing apparatus according to the fourth embodiment adjusts the capacity of the storing container, based on the filling amount.

Accordingly, by using a capacity-changeable storing container (for example, a cell storing bag), the manufacturing apparatus according to the fourth embodiment can manufacture a cell stock with a storing container of identical standards (for example, a quality of material, and a size before a capacity change) even in a case where the filling amount is different.

Conventionally, as described above, the lineup of volumes of cell storing containers is not so designed as to cover freely selected capacities. In a case of filling with use of a cell storing bag that is relatively adaptable to a freely selected capacity, compared to a cryotube or the like, cell-containing products with different thicknesses in accordance with filling amounts are manufactured. In a case of freezing them at a time of keeping, it is considered that there is a possibility that a difference in thickness between cell suspensions adversely affects a survival rate of cells. Since it is considered that a breakage of a cell membrane due to the behavior of a water crystal occurring in a cell at a time of freeze-keeping affects a survival rate of cells after freeze-thaw, it is necessary to strictly manage the cooling temperature at a time of freezing in order to increase the survival rate of cells. For this purpose, it is considered that cell stocks made of cell-containing products with uniform thickness, rather than cell-containing products with different thicknesses, are easy to treat in regard to management of cooling temperatures.

### Fifth Embodiment

In the fourth embodiment, the method of adjusting the capacity of the variable container was described as a concrete supply method of the storing container by the container supply unit 22. In a fifth embodiment, a method of selecting an optimal storing container from among a plurality of different storing containers is described as a concrete supply method of the storing container by the container supply unit 22.

In the fifth embodiment, by the control function 83, the control circuitry 8 selects a container corresponding to a filling amount. After selecting the container, the control circuitry 8 causes the container supply unit 22 to output the storing container corresponding to the filling amount as the selected container.

In the fifth embodiment, in accordance with control of the control circuitry 8, the container supply unit 22 outputs the selected container that is the storing container corresponding to the filling amount.

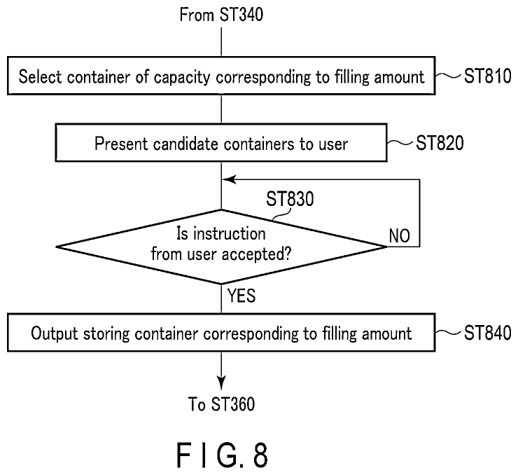

FIG. 8 is a flowchart exemplarily illustrating a supply method of a storing container of a manufacturing apparatus according to the fifth embodiment. The flowchart of FIG. 8 is a concrete example of step ST350 of the flowchart of FIG. 3. The flowchart of FIG. 8 starts after the process of step ST340 of FIG. 3.

### (Step ST810)

After accepting an instruction from the user in step ST340, the control circuitry 8 selects, by the control function 83, a container of a capacity corresponding to a filling amount. Specifically, based on the information of the filling amount, the control circuitry 8 selects an optimal container for the determined filling amount. The control circuitry 8 may select a plurality of containers having different sizes. Besides, selected containers may be containers of different qualities of material and of different specifications, and may be, for example, a vial bottle, a cryotube, and a cell storing bag.

### (Step ST820)

The control circuitry 8 presents candidate containers to the user by the control function 83. Specifically, the control circuitry 8 causes a display functioning as the output interface 5 to display candidate containers.

### (Step ST830)

The control circuitry 8 determines, by the control function 83, whether an instruction from the user is accepted. The instruction from the user is an instruction for selecting a container to be filled, from among the presented candidate containers. If the instruction from the user is accepted, the process advances to step ST840. If the instruction from the user is not accepted, the process stands by in step ST340 until accepting an instruction from the user.

### (Step ST840)

After accepting the instruction from the user, the control circuitry 8 outputs the information of the selected container to the container supply unit 22. The container supply unit 22 outputs the storing container corresponding to the filling amount, which is the selected container. After step ST840, the process advances to step ST360 of FIG. 3.

As has been described above, the manufacturing apparatus according to the fifth embodiment selects the optimal storing container from among the storing containers having different sizes, based on the filling amount, at the time of supplying the storing container.

Accordingly, since the manufacturing apparatus according to the fifth embodiment can select the optimal storing container from among the storing containers that are different with respect to a size, a quality of material and specifications, an optimal cell stock can be manufactured regardless of the kind of cells.

### Another Embodiment

In each of the above-described embodiments, the manufacture of cell stocks was described, but the embodiments are not limited to this. For example, the embodiments may be applied to the manufacture of cell processing products (products of regenerative medicine or the like, and specific cell processing products) that are applied to patients. Specifically, the manufacturing apparatus 1 determines a filling amount of a cell processing product in accordance with cell information, a cell number per body weight of 1 kg that is necessary for administration to a patient, and the body weight of the patient, and fills the cell processing product in a storing container corresponding to the filling amount.

In the case of treating the cell processing product, the manufacturing apparatus 1 may supply the storing container corresponding to the filling amount, by also taking into account the information of the convenience of administration to a patient, or the like. For example, in the case of supplying the storing container corresponding to such a filling amount of the cell processing product as to be administered to the patient multiple times at time intervals, the manufacturing apparatus 1 may fill the cell processing product into one storing container, or may fill the cell processing product dividedly into a plurality of storing containers. The reason why the cell processing product is dividedly filled in the storing containers relates to, for example, a storage period at a time of normal temperatures. Since storage containers are basically frozen and kept, in a case where a storage period at normal temperatures after unfreezing is long, cell processing products for multiple-time administrations may be stored in one storing container. On the other hand, in a case where a storage period at normal temperatures after unfreezing is short, cell processing products for multiple-time administrations may be stored in a plurality of storing containers so that a cell processing product for one-time administration can be unfrozen each time.

In another embodiment, the control circuitry 8 acquires processing product information and body weight information by the acquisition function 81. The processing product information relates to a cell processing product, and includes, for example, information relating to a storage method and an administration method. The body weight information is information of the body weight of a patient or the like (target person) to which the cell processing product is administered. The control circuitry 8 determines, by the determination function 82, the filling amount of the cell processing product, based on the processing product information and the body weight information. By the control function 83, the control circuitry 8 causes the container supply unit 22 to supply a storing container for storing the cell processing product of the determined filling amount. In addition, the control circuitry 8 causes the cell liquid filling unit 23 to fill the cell processing product of the determined filling amount into the supplied storing container.

In the another embodiment, in accordance with control of the control circuitry 8, the container supply unit 22 supplies the storing container for storing the cell processing product of the determined filling amount. In addition, in accordance with control of the control circuitry 8, the cell liquid filling unit 23 fills the cell processing product of the determined filling amount into the supplied storing container.

As has been described above, in regard to the cell processing product as the cell suspension, like each of the above-described embodiments, the manufacturing apparatus according to the another embodiment determines the filling amount of the cell processing product, supplies the storing container corresponding to the filling amount, and fills the cell processing product of the filling amount into the storing container.

Accordingly, the manufacturing apparatus according to the another embodiment can fill the cell processing product into the appropriate storing container, regardless of the kind of cell processing product.

For example, in a case of manufacturing a product of regenerative medicine or the like by using the present manufacturing apparatus, the user can avoid additional work, such as additional expansion culture and passage, on a stable manufacturing schedule. In another aspect, a patient who receives the provision of the product of regenerative medicine or the like can receive the administration (or adaptation) of this product at a proper timing.

Note that the manufacturing apparatus according to the present embodiment can establish an iPS cell at a time of primary culture, and can be used in manufacturing a cell stock with use of the iPS cell.

### Sixth Embodiment

In the first to fifth embodiments, the description was given of the manufacturing apparatus that manufactures the cell stock by determining the filling amount of the cell suspension corresponding to one cell stock, and filling the cell suspension of the determined filling apparatus into the storing container. On the other hand, in a sixth embodiment, a description is given of an information processing apparatus that determines a filling amount of a cell suspension, without proceeding up to the manufacture of a cell stock.

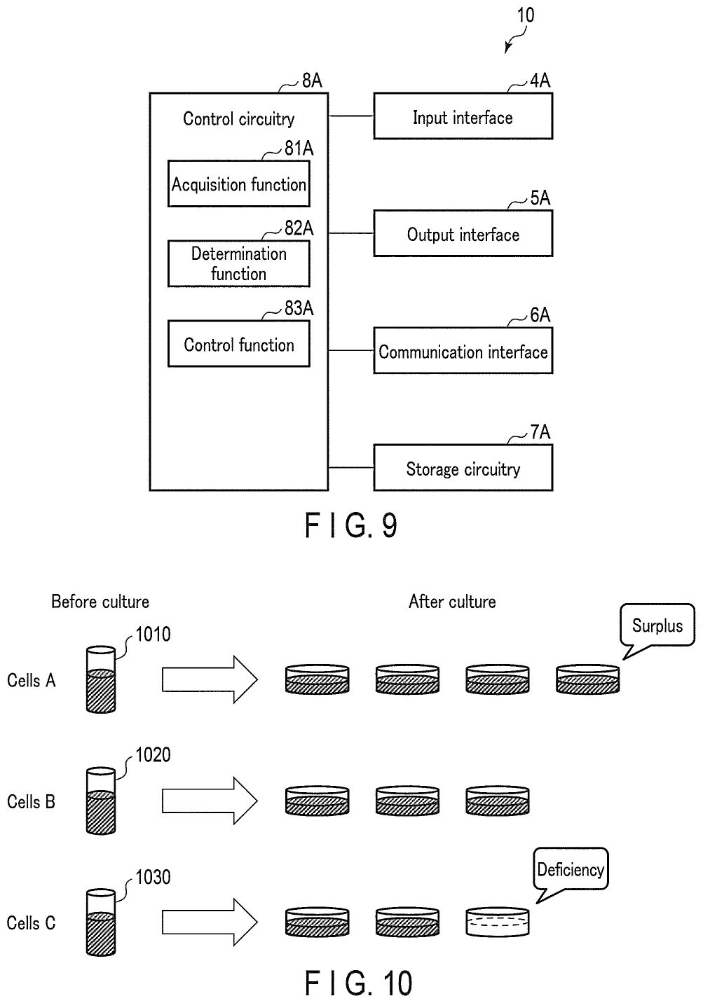

FIG. 9 is a diagram illustrating a configuration example of an information processing apparatus 10 according to the sixth embodiment. The information processing apparatus 10 includes an input interface 4A, an output interface 5A, a communication interface 6A, storage circuitry 7A, and control circuitry 8A. Note that since the input interface 4A, output interface 5A, communication interface 6A and storage circuitry 7A are similar to the input interface 4, output interface 5, communication interface 6 and storage circuitry 7 of the manufacturing apparatus 1, a description thereof is omitted.

The control circuitry 8A is a processor functioning as a central unit of the information processing apparatus 10. For example, by executing a processing program stored in the storage circuitry 7A, the control circuitry 8A implements functions corresponding to this processing program. Specifically, the control circuitry 8A includes an acquisition function 81A (acquisition unit), a determination function 82A (determination unit) and a control function 83A (control unit) by executing the processing program. Note that in the present embodiment, a case is described in which the acquisition function 81A, determination function 82A and control function 83A are implemented by a single processor, but the embodiment is not limited to this. For example, a plurality of independent processors may be combined to constitute control circuitry, and the respective processors may execute the processing program, thereby implementing the acquisition function 81A, determination function 82A and control function 83A. Besides, since the acquisition function 81A and determination function 82A are similar to the acquisition function 81 and determination function 82 of the control circuitry 8 in the manufacturing apparatus 1, a description thereof is omitted.

By the control function 83A, the control circuitry 8A presents a determined filling amount, or candidate filling amounts, on a display functioning as the output interface 5A.

The configuration of the information processing apparatus 10 according to the sixth embodiment has been described. Since the operation of the information processing apparatus according to the sixth embodiment is substantially similar to the operation of steps ST310 to ST330 of FIG. 3, a description thereof is omitted.

As has been described above, the information processing apparatus according to the sixth embodiment acquires information relating to proliferation characteristics at a time of cell culture of cells included in a cell suspension, and determines a filling amount of the cell suspension corresponding to one cell stock, based on the information relating to the proliferation characteristics.

Accordingly, the information processing apparatus according to the sixth embodiment can make a cell number after expansion culture substantially equal to a target cell number, by considering a variance in proliferation characteristics, in regard to the cell suspension that is filled in the cell stock. Thus, the information processing apparatus according to the sixth embodiment can be utilized for the manufacture of a cell stock in which proliferation characteristics of cells are taken into account.

According to at least one of the above-described embodiments, a cell stock in which proliferation characteristics of cells are taken into account can be manufactured.

Note that the term “processor” used in the above description means, for example, a CPU (Central Processing Unit), a GPU (Graphics Processing Unit), or circuitry such as an application specific integrated circuit (ASIC) or a programmable logic device (for example, simple programmable logic device (SPLD), a complex programmable logic device (CPLD) or a field programmable gate array (FPGA)). If the processor is, for example, a CPU, the processor implements the functions by reading and executing the program stored in the storage circuitry. On the other hand, if the processor is, for example, an ASIC, the functions are directly incorporated in the circuitry of the processor as logic circuitry, instead of the program being stored in the storage circuitry. Note that, aside from the case where each of the processors of the embodiments is constructed as single circuitry for each processer, the processors may be constructed as a single processor by combining a plurality of independent circuits and thereby the functions may be implemented. Furthermore, a plurality of structural elements in the drawings may be integrated into a single processor, and the functions thereof may be implemented.

In addition, each of the functions according to the embodiments can also be implemented by installing the programs that execute the above-described process into a computer such as a workstation, and loading the programs on the memory. At this time, the programs that can cause the computer to execute the above-described methods can be distributed by being stored in storage media such as a magnetic disk (hard disk or the like), an optical disc (CD-ROM, DVD or the like), a semiconductor memory or the like.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

In connection with the above-described embodiments, the following supplementary notes are disclosed as aspects and selective features of the invention.

(1) A manufacturing apparatus comprises processing circuitry configured to:
acquire information relating to proliferation characteristics of cells when using a cell suspension;
determine a filling amount of the cell suspension corresponding to one housing container, based on the information relating to the proliferation characteristics; and
fill the cell suspension of the filling amount into the housing container for containing the cell suspension of the filling amount.

(2) The information relating to the proliferation characteristics may include a proliferation rate of the cells, and the processing circuitry may further determine the filling amount, based on the proliferation rate.

(3) The information relating to the proliferation characteristics may include a doubling time of the cells, and the processing circuitry may further determine the filling amount, based on the doubling time.

(4) The information relating to the proliferation characteristics may include a proliferation curve of the cells, and the processing circuitry may further determine the filling amount, based on the proliferation curve.

(5) The information relating to the proliferation characteristics may include first proliferation characteristic information at a time of primary culture of the cells, and second proliferation characteristic information after the cells are frozen and unfrozen, and the processing circuitry may further determine the filling amount, based on the first proliferation characteristic information and the second proliferation characteristic information.

(6) The information relating to the proliferation characteristics may include sample information on a sample from which the cells originate, and the processing circuitry may further determine the filling amount, based on the sample information.

(7) The manufacturing apparatus may further include a supply unit configured to supply the housing container.

(8) The supply unit may fabricate the housing container corresponding to the filling amount.

(9) The supply unit may fabricate the housing container by blow molding.

(10) The supply unit may adjust a capacity of the housing container, based on the filling amount.

(11) The supply unit may select the housing container from among a plurality of housing containers, based on the filling amount.

(12) The housing containers may different with respect to at least one of a size, a quality of material, and specifications.

(13) The manufacturing apparatus may include a control unit configured to present the filling amount to an operator, wherein the supply unit may supply the housing container by using an instruction from the operator as a trigger.

(14) The processing circuitry may further acquire suspension information relating to the cell suspension, and a target condition including a cell culture period and a target cell number of the cells, and determine the filling amount, based on the information relating to the proliferation characteristics, the suspension information, and the target condition.

(15) The information relating to the proliferation characteristics may include a doubling time of the cells, and the processing circuitry may determine the filling amount, based on the doubling time, the suspension information and the target condition.

(16) The suspension information may include at least one of a total amount of the cell suspension, and a living cell number and a cell concentration in the cell suspension.

(17) The cell suspension may be a cell processing product, and the processing circuitry may further acquire processing product information relating to the cell processing product, and body weight information of a target person to which the cell processing product is administered, and determine the filling amount, based on the processing product information and the body weight information.

(18) An information processing apparatus comprises processing circuitry configured to:
acquire information relating to proliferation characteristics of cells when using a cell suspension; and
determine a filling amount of the cell suspension corresponding to one housing container, based on the information relating to the proliferation characteristics.

(19) A manufacturing method comprising:
acquiring information relating to proliferation characteristics of cells when using a cell suspension;
determining a filling amount of the cell suspension corresponding to one housing container, based on the information relating to the proliferation characteristics; and
filling the cell suspension of the filling amount into the housing container for containing the cell suspension of the filling amount.

What is claimed is:

1. A manufacturing apparatus comprising processing circuitry configured to:
acquire information relating to proliferation characteristics of cells when using a cell suspension;
determine a filling amount of the cell suspension corresponding to one housing container, based on the information relating to the proliferation characteristics; and
fill the cell suspension of the filling amount into the housing container for containing the cell suspension of the filling amount.

2. The manufacturing apparatus according to claim 1, wherein the information relating to the proliferation characteristics includes a proliferation rate of the cells, and the processing circuitry is further configured to determine the filling amount, based on the proliferation rate.

3. The manufacturing apparatus according to claim 1, wherein the information relating to the proliferation characteristics includes a doubling time of the cells, and the processing circuitry is further configured to determine the filling amount, based on the doubling time.

4. The manufacturing apparatus according to claim 1, wherein the information relating to the proliferation characteristics includes a proliferation curve of the cells, and the processing circuitry is further configured to determine the filling amount, based on the proliferation curve.

5. The manufacturing apparatus according to claim 1, wherein the information relating to the proliferation characteristics includes first proliferation characteristic information at a time of primary culture of the cells, and second proliferation characteristic information after the cells are frozen and unfrozen, and the processing circuitry is further configured to determine the filling amount, based on the first proliferation characteristic information and the second proliferation characteristic information.

6. The manufacturing apparatus according to claim 1, wherein the information relating to the proliferation characteristics includes sample information on a sample from which the cells originate, and the processing circuitry is further configured to determine the filling amount, based on the sample information.

7. The manufacturing apparatus according to claim 1, further comprising a supply unit configured to supply the housing container.

8. The manufacturing apparatus according to claim 7, wherein the supply unit is configured to fabricate the housing container corresponding to the filling amount.

9. The manufacturing apparatus according to claim 8, wherein the supply unit is configured to fabricate the housing container by blow molding.

10. The manufacturing apparatus according to claim 7, wherein the supply unit is configured to adjust a capacity of the housing container, based on the filling amount.

11. The manufacturing apparatus according to claim 7, wherein the supply unit is configured to select the housing container from among a plurality of housing containers, based on the filling amount.

12. The manufacturing apparatus according to claim 11, wherein the housing containers are different with respect to at least one of a size, a quality of material, and specifications.

13. The manufacturing apparatus according to claim 7, further comprising a control unit configured to present the filling amount to an operator, wherein the supply unit is configured to supply the housing container by using an instruction from the operator as a trigger.

14. The manufacturing apparatus according to claim 1, wherein the processing circuitry is further configured to:
acquire suspension information relating to the cell suspension, and a target condition including a cell culture period and a target cell number of the cells; and
determine the filling amount, based on the information relating to the proliferation characteristics, the suspension information, and the target condition.

15. The manufacturing apparatus according to claim 14, wherein the information relating to the proliferation characteristics includes a doubling time of the cells, and the processing circuitry is further configured to determine the filling amount, based on the doubling time, the suspension information and the target condition.

16. The manufacturing apparatus according to claim 14, wherein the suspension information includes at least one of a total amount of the cell suspension, and a living cell number and a cell concentration in the cell suspension.

17. The manufacturing apparatus according to claim 1, wherein the cell suspension is a cell processing product, and the processing circuitry is further configured to:
acquire processing product information relating to the cell processing product, and body weight information of a target person to which the cell processing product is administered; and
determine the filling amount, based on the processing product information and the body weight information.

18. An information processing apparatus comprising processing circuitry configured to:
acquire information relating to proliferation characteristics of cells when using a cell suspension; and
determine a filling amount of the cell suspension corresponding to one housing container, based on the information relating to the proliferation characteristics.

19. A manufacturing method comprising:
acquiring information relating to proliferation characteristics of cells when using a cell suspension;
determining a filling amount of the cell suspension corresponding to one housing container, based on the information relating to the proliferation characteristics; and
filling the cell suspension of the filling amount into the housing container for containing the cell suspension of the filling amount.
